(19) United States
(12) Patent Application Publication
(10) Pub. No.: US 2010/0298521 A1
(43) Pub. Date: Nov. 25, 2010

(54) PROCESS FOR PRODUCING (POLY)THIOL COMPOUND FOR USE AS OPTICAL MATERIAL, AND POLYMERIZABLE COMPOSITION CONTAINING THE COMPOUND

(75) Inventors: Shigetoshi KUMA, Kurume-shi (JP); Michiharu SAKATA, Omuta-shi (JP); Seiichi KOBAYASHI, Omuta-shi (JP)

Correspondence Address: BUCHANAN, INGERSOLL & ROONEY PC, POST OFFICE BOX 1404, ALEXANDRIA, VA, 22313-1404, US

(73) Assignee: MITSUI CHEMICALS, INC., Minato-ku (JP)

(21) Appl. No.: 12/838,659

(22) Filed: Jul. 19, 2010

Related U.S. Application Data
(63) Continuation of application No. 12/297,265, filed on Oct. 15, 2008.

(30) Foreign Application Priority Data
Apr. 19, 2006 (JP) 2006-115289

(51) Int. Cl. C08G 18/72; C07C 319/08

(52) U.S. Cl. 528/85; 568/66

(57) ABSTRACT

A process for producing a (poly)thiol compound for an optical material. The process involves reacting an organic (poly)halogen compound or a (poly)alcohol compound with thiourea to produce an isothiuronium salt and hydrolyzing the isothiuronium salt in the presence of ammonia water to thereby produce the (poly)thiol compound, wherein the thiourea has a calcium content of not more than 1.0 wt %.

### TECHNICAL FIELD

The present application is a Continuation Application of U.S. application Ser. No. 12/297,265, filed Oct. 15, 2008, which is the National Stage of International Application No. PCT/JP2007/000399, filed Apr. 12, 2007, and claims foreign priority to Japanese Application No. 2006-115289, filed Apr. 19, 2006, the entire contents of each of which are incorporated by reference herein.

The present invention relates to a (poly)thiol compound for an optical material and a process for producing the same, and a polymerizable composition composed of the (poly)thiol compound and a polyiso(thio)cyanate compound, which is used as an optical material of a polyurethane based lens or the like exhibiting excellent optical properties.

### BACKGROUND ART

As a method for the production of a thiol compound, many methods have been known from the past. As the method, there can be exemplified, for example, a method including reducing a disulfide compound, a method including reacting an organic halide with an alkali metal hydrosulfide salt or an alkali metal sulfide salt such as sodium hydrosulfide, potassium hydrosulfide or the like, a method including reacting an organic halide or alcohol with thiourea to produce an isothiuronium salt and hydrolyzing the isothiuronium salt with a base, a method including producing a Bunte salt, a method including producing dithiocarbamic acid ester, a method including using a Grignard reagent and sulfur, a method including fragmentizing a C—S bond of sulfide, a method including ring-opening episulfide, a method including reacting a compound having a carbonyl group as a starting material with hydrogen sulfide, a method including adding hydrogen sulfide or thioacetic acid to alkene, and the like.

Of the methods, the method for the production of a thiol compound by producing an isothiuronium salt from an organic halide or alcohol brings a high yield, produces a small amount of by-product, is excellent in operability, results in obtaining a product with good quality in many cases as compared to other production methods. Therefore, this method is one of methods for the production of a thiol compound which is generally used as the best method,

Furthermore, for the reaction of an organic (poly)halogen compound or a (poly)alcohol compound with thiourea, it is known that a method for the production of a (poly)thiol compound by adding sulfuric acid to produce an isothiuronium salt is capable of effectively producing a (poly)thiol compound in a high yield and at low cost (refer to Patent Document 1).

At that time, thiourea in use is produced from lime nitrogen and hydrogen sulfide, or calcium hydrosulfide. Further, it is known that a thiourea-containing solution is purified by a strong basic ion exchange resin (refer to Patent Document 2).

Furthermore, there has been described that a poly(thio)urethane resin obtained by reacting a (poly)thiol compound obtained by this production method with a polyiso(thio)cyanate compound is colorless and transparent, has a high refractive index and a low dispersion, is excellent in impact resistance, dyeing property, processability and the like, and is one of resins which are optimum for plastic lenses of optical materials (refer to Patent Documents 3, 4 and 5).

However, the (poly)thiol compound obtained even in the above production method caused a problem of coloring in many times and was difficult to be stably produced.

Patent Document 1: Japanese Patent Laid-open No. 2001-39944

Patent Document 2: Japanese Patent Laid-open No. S48 (1973)-49722

Patent Document 3: Japanese Patent Laid-open No. H9 (1997)-110955

Patent Document 4: Japanese Patent Laid-open No. H9 (1997)-110956

Patent Document 5: Japanese Patent Laid-open No. H7 (1995)-252207

### DISCLOSURE OF THE INVENTION

However, a (poly)thiol compound produced by the conventional methods including producing an isothiuronium salt caused a problem of coloring, or a poly(thio)urethane resin obtained by using the (poly)thiol compound caused a problem of coloring or whitening in some cases.

For that reason, there has been demanded that the occurrence of such problems in the methods including producing an isothiuronium salt should be suppressed to the utmost, and an industrial method for the production of a (poly)thiol compound without causing coloring should be developed. Accordingly, a plastic lens composed of a poly(thio)urethane resin without causing coloring or whitening needed to be provided to the world in a stable manner.

The present invention relates to a process for producing a (poly)thiol compound by reacting an organic (poly) halogen compound or a (poly)alcohol compound with thiourea to produce an isothiuronium salt and hydrolyzing the obtained isothiuronium salt, and a process for producing a colorless and transparent (poly)thiol compound in which coloring is suppressed. Furthermore, the invention is to provide, by polymerizing the (poly)thiol compound obtained by the process of the present invention with a polyiso(thio)cyanate compound, a colorless and transparent poly(thio)urethane resin in which coloring or whitening is suppressed, and a plastic lens which is useful as an optical material.

In order to solve the above objects, the present inventors have conducted an extensive study and as a result, have confirmed that coloring of a poly(thio)urethane resin is caused by the color tone of a (poly)thiol compound in use. Furthermore, to search for the cause of coloring of a (poly)thiol compound, the inventors have conducted an extensive study on a process for producing a (poly)thiol compound prepared by producing an isothiuronium salt from an organic (poly) halogen compound or a (poly) alcohol compound and hydrolyzing the isothiuronium salt, and the production conditions. As a result, the inventors have found conditions for the production of a colorless and transparent (poly)thiol compound in which coloring is suppressed. However, even if the production conditions were the same, the (poly)thiol compound was colored in some cases and was difficult to be stably produced.

Even though the production conditions were the same, to solve the aforementioned problem of coloring of the (poly)thiol compound, they have checked in detail the quality of thiourea used for producing an isothiuronium salt. They have continued an extensive study on how the purity of thiourea and the quality of a trace of impurities contained in thiourea have influence on coloring of the obtained (poly)thiol, and whitening or coloring of the poly(thio)urethane resin. As a result, surprisingly, when the amount of impurities contained in thiourea is not less than a specific amount, coloring of the obtained (poly)thiol compound has been clearly observed. They have conducted an extensive study on the specification of the impurities and as a result, have specified that a main ingredient of the impurities is calcium. As a result, they have found that, when a (poly)thiol compound is produced with thiourea having a calcium content of not more than a specific amount as a starting material, a colorless and transparent (poly)thiol compound in which coloring is suppressed can be stably obtained. Furthermore, they have found that a colorless and transparent poly(thio)urethane resin in which coloring and whitening are suppressed by using the compound is obtained. Thus, the present invention has been completed.

That is, the present invention is specified by the following matters:

(1) a process for producing a (poly)thiol compound for an optical material comprising:

reacting an organic (poly)halogen compound or a (poly) alcohol compound with thiourea to produce an isothiuronium salt, and

hydrolyzing the obtained isothiuronium salt to produce a (poly)thiol compound,

in which the calcium content in the thiourea is not more than 1.0 wt %;

(2) the process for producing a (poly)thiol compound for an optical material as set forth in (1) above, in which the (poly)thiol compound has a sulfur atom in addition to a thiol group;

(3) the process for producing a (poly)thiol compound for an optical material as set forth in (2) above, in which the (poly)thiol compound having a sulfur atom in addition to a thiol group has one or two or more kinds selected from the group consisting of 1,2-bis[(2-mercaptoethyl)thio]-3-mercaptopropane, 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients;

(4) a polymerizable composition containing the (poly)thiol compound for an optical material produced by the process as set forth in any one of (1) to (3) above and a polyiso(thio)cyanate compound;

(5) a resin obtained by curing the polymerizable composition as set forth in (4) above;

(6) an optical material containing the resin as set forth in (5) above; and

(7) a lens containing the resin as set forth in (5) above.

In the above (6) and (7), a phrase “containing the resin” refers to both a case in which the entire optical material or the entire lens is composed of the resin and a case in which a part of the optical material or the lens is composed of the resin.

The process for producing a (poly)thiol compound for an optical material of the present invention is suitable for an industrial application, and is capable of stably obtaining a colorless and transparent (poly)thiol compound in which coloring is suppressed. The poly(thio)urethane resin obtained by using a polymerizable composition composed of a (poly)thiol compound for an optical material obtained in accordance with the production process of the present invention and a polyiso(thio)cyanate compound is colorless and transparent, in which coloring and whitening are suppressed. According to the present invention, it is possible to provide a colorless and transparent polyurethane based lens useful as an optical material and a transparent material in a stable manner which contribute to the development of the related fields.

### BEST MODE FOR CARRYING OUT THE INVENTION

The present invention will be illustrated in detail below.

The present invention relates to a process for producing a (poly)thiol compound for an optical material by reacting an organic (poly)halogen compound or a (poly)alcohol compound with thiourea to produce an isothiuronium salt and hydrolyzing the obtained isothiuronium salt. The content of calcium in thiourea used for the present invention is not more than a specific amount. That is, thiourea having a calcium content of not more than 1.0 wt % is used.

Thiourea to be used as a starting material for forming an isothiuronium salt is mainly produced by reacting lime nitrogen with hydrogen sulfide. Examples of impurities contained in thiourea include unreacted lime nitrogen, and further by-produced calcium hydroxide. That is, when calcium is contained in thiourea in excess of a specific amount, the obtained (poly)thiol compound is colored, and a polymerizable composition obtained by mixing with a polyiso(thio)cyanate compound and the obtained resin are colored or whitened.

The calcium content in thiourea used for the present invention is preferably from 0.0005 to 1.0 wt %, more preferably from 0.0005 to 0.5 wt % both inclusive, and further preferably from 0.0005 to 0.2 wt % both inclusive from the viewpoint of suppression of coloring and whitening.

When the calcium content is not more than 1.0 wt %, a (poly)thiol compound produced by using the thiourea is colorless and transparent, in which coloring is suppressed. Further, the poly(thio)urethane resin obtained by polymerizing the produced (poly)thiol compound with polyiso(thio)cyanate composes a colorless and transparent poly(thio)urethane based lens in which whitening and coloring are suppressed.

The calcium content is measured in the following manner. Thiourea is made into an aqueous solution, and then its calcium content is quantitatively analyzed by an ion chromatographic method.

The calcium content can be reduced by employing a method such as purification, acid treatment, recrystallization or the like, and can be not more than 1.0 wt %. Specifically, the calcium content can be reduced, for example, by acid treatment using hydrochloric acid, sulfuric acid or the like, and can also be reduced by a recrystallization method using an aqueous system.

The organic (poly)halogen compound as the other starting material is a compound having one or more halogen atoms in a molecule, and is not particularly restricted in terms of quality.

Concrete examples of the starting material organic (poly)halogen compound include bis(2,3-dichloropropyl)sulfide, 1,1,1-tris(chloromethyl)propane, 1,1,1-tris(bromomethyl)propane, 1,2-bis(2-chloroethylthio)-3-chloropropane, 1,2-bis(2-bromoethylthio)-3-bromopropane, 1,3-bis(2-chloroethylthio)-2-chloropropane, 1,3-bis(2-bromoethylthio)-2-bromopropane, 2,5-bis(chloromethyl)-1,4-dithiane, 2,5-bis(bromomethyl)-1,4-dithiane, 4,8-dichloromethyl-1,11-dichloro-3,6,9-trithiaundecane, 4,8-dichloromethyl-1,11-dichloro-3,6,9-trithiaundecane, 5,7-dichloromethyl-1,11-dichloro-3,6,9-trithiaundecane, 4,8-dibromomethyl-1,11-dibromo-3,6,9-trithiaundecane, 4,7-dibromomethyl-1,11-dibromo-3,6,9-trithiaundecane, 5,7-dibromomethyl-1,11-dibromo-3,6,9-trithiaundecane, 1,5,9,13-tetrachloro-3,7,11-trithiamidecane, 1,5,9,13-tetrabromo-3,7,11-trithiamidecane, 1,2,6,7-tetrachloro-4-thiaheptane, 1,2,6,7-tetrabromo-4-thiaheptane and the like, but the present invention is not restricted to these exemplified compounds.

The (poly)alcohol compound as the other starting material is a compound having one or more hydroxy groups in a molecule, and is not particularly restricted in terms of quality. Concrete examples thereof include bis(2,3-dihydroxy)sulfide, 1,1,1-tris(hydroxymethyl)propane, 1,2-bis(2-hydroxyethylthio)-3-hydroxypropane, 1,3-bis(2-hydroxyethylthio)-2-hydroxypropane, 2,5-bis(hydroxymethyl)-1,4-dithiane, 4,8-dihydroxymethyl-1,11-dihydroxy-3,6,9-trithiaundecane, 4,7-dihydroxymethyl-1,11-dihydroxy-3,6,9-trithiaundecane, 5,7-dihydroxymethyl-1,11-dihydroxy-3,6,9-trithiaundecane, 1,5,9,13-tetrahydroxy-3,7,11-trithiamidecane, 1,2,6,7-tetrahydroxy-4-thiaheptane, pentaerythritol and the like, but the present invention is not restricted to these exemplified compounds.

In the present invention, a process including reacting an organic (poly)halogen compound or a (poly)alcohol compound with thiourea is preferably carried out in a solvent. The solvent used at that time is, for example, water, alcohol other than a starting material or an organic halogen compound.

As alcohol, for example, methanol, ethanol, isopropanol, butanol, methoxyethanol and the like are preferably used.

Examples of the organic halogen compound include dichloromethane, dichloroethane, chloroform, chlorobenzene, o-dichlorobenzene, p-dichlorobenzene and the like.

Hydrolysis which subsequently carried out after producing an isothiuronium salt is conducted by using usual base water, similar to a conventional method. Examples of the kind of base water in use include base water such as sodium hydroxide water, potassium hydroxide water, ammonia water, hydrazine water, sodium carbonate water and the like. Of these, when ammonia water is used, particularly preferable results are presented.

The amount of the base used is generally in the range of 1.0 to 3.0 equivalents both inclusive for obtaining the preferable results, and in the range of 1.0 to 2.0 equivalents both inclusive for obtaining the further preferable results, based on the number of halogen atoms bonded to the organic halogen compound or the amount of hydrohalogenated acid which is well used in case of (poly) alcohols.

Since the reaction temperature at the time of hydrolysis is different depending on the kind of base water in use, the reaction temperature is difficult to be restricted, but it is generally in the range of 0 to 100 degree centigrade, and preferably in the range of 20 to 70 degree centigrade.

As the solvent used for hydrolysis, there are preferably used, for example, water; alcohols such as methanol, ethanol, isopropanol, butanol, methoxyethanol and the like; aromatic hydrocarbon solvents such as toluene, xylene and the like; and halogen solvents such as chlorobenzene, dichlorobenzene and the like.

The reaction for producing an isothiuronium salt in the prior step is carried out in a water solvent and the isothiuronium salt may be subjected to hydrolysis as it is without taking out a reactant. In that case, an aromatic hydrocarbon solvent such as toluene, xylene or the like is added to the reaction system for carrying out hydrolysis in a double-layer system. In such a process, the generated (poly)thiol compound is extracted to an organic solvent, whereby washing procedures carried out thereafter are conducted effectively and within a short period of time in some cases; therefore, it is preferable.

The thus-obtained reaction solution containing such a (poly)thiol compound in the present invention is usually subjected, if necessary, to various washing treatments including acid washing, base washing, water washing or the like for removing the solvent and then filtering to obtain as a product. Furthermore, the solution may be purified by other various purification methods such as distillation, column chromatography, recrystallization or the like.

According to the production process of the present invention, a colorless and transparent (poly)thiol compound in which coloring is suppressed is obtained. The (poly)thiol compound obtained in the present invention may have a sulfur atom in addition to a thiol group. Specifically, for example, in the following compounds, an effect of the present invention is more remarkably obtained.

Examples thereof include (poly)thiol compounds having main ingredients of one or two or more kinds selected from the group consisting of 1,2-bis[(2-mercaptoethyl)thio]-3-mercaptopropane, 4,8-dimercaptomethyl-1,11-dimercapto-3,6,9-trithiaundecane, 4,7-dimercaptomethyl-1,11-dimercapto-3,6,9-trithiaundecane, 5,7-dimercaptomethyl-1,11-dimercapto-3,6,9-trithiaundecane, 2,5-dimercaptomethyl-1,4-dithiane, 1,1,3,3-tetramercaptomethyl-2-thiapropane, bis(2,3-dimercaptopropyl)sulfide, 1,1,1-tris(mercaptomethyl)propane, 1,5,9,13-tetramercapto-3,7,11-trithiamidecane, tetramercaptomethylmethane and the like, but the present invention is not restricted to these exemplified compounds.

The polyiso(thio)cyanate compound used in the present invention is a compound having at least two or more iso(thio)cyanate groups in a molecule, and is not particularly limited. Concrete examples thereof include aliphatic polyisocyanate compounds such as hexamethylene diisocyanate, 2,2-dimethylpentane diisocyanate, 2,2,4-trimethylhexane diisocyanate, butene diisocyanate, 1,3-butadiene-1,4-diisocyanate, 2,4,4-trimethylhexamethylene diisocyanate, 1,6,11-undecanetriisocyanate, 1,3,6-hexamethylene triisocyanate, 1,8-diisocyanato-4-isocyanatomethyloctane, bis(isocyanatoethyl)carbonate, bis(isocyanatoethyl)ether, lysine diisocyanatomethyl ester, lysine triisocyanate and the like;

polyisocyanate compounds having an aromatic compound such as xylylene diisocyanate, 1,2-diisocyanatobenzene, 1,3-diisocyanatobenzene, 1,4-diisocyanatobenzene, 2,4-diisocyanatotoluene, ethylphenylene diisocyanate, isopropylphenylene diisocyanate, dimethylphenylene diisocyanate, diethylphenylene diisocyanate, diisopropylphenylene diisocyanate, trimethylbenzene triisocyanate, benzene triisocyanate, biphenyl diisocyanate, toluidine diisocyanate, 4,4′-methylenebis(phenyl isocyanate), 4,4′-methylenebis(2-methylphenyl isocyanate), bibenzyl-4,4′-diisocyanate, bis(isocyanatophenyl)ethylene, bis(isocyanatoethyl)benzene, bis(isocyanatopropyl)benzene, α,α,α′,α′-tetramethylxylylene diisocyanate, bis(isocyanatobutyl)benzene, bis(isocyanatomethyl)naphthalene, bis(isocyanatomethylphenyl)ether, bis(isocyanatoethyl)phthalate, 2,6-di(isocyanatomethyl)furan and the like;

sulfur-containing aliphatic polyisocyanate compounds such as bis(isocyanatomethyl)sulfide, bis(isocyanatoethyl)sulfide, bis(isocyanatopropyl)sulfide, bis(isocyanatohexyl)sulfide, bis(isocyanatomethyl)sulfone, bis(isocyanatomethyl)disulfide, bis(isocyanatoethyl)disulfide, bis(isocyanatopropyl)disulfide, bis(isocyanatomethylthio)methane, bis(isocyanatoethylthio)methane, bis(isocyanatomethylthio)ethane, bis(isocyanatoethylthio)ethane, 1,5-diisocyanato-2-isocyanatomethyl-3-thiapentane, 1,2,3-tris(isocyanatomethylthio)propane, 1,2,3-tris(isocyanatoethylthio)propane, 3,5-dithia-1,2,6,7-heptane tetraisocyanate, 2,6-diisocyanatomethyl-3,5-dithia-1,7-heptane diisocyanate, 2,5-diisocyanate methyl thiophene, 4-isocyanatoethylthio-2,6-dithia-1,8-octane diisocyanate and the like;

aromatic sulfide based polyisocyanate compounds such as 2-isocyanatophenyl-4-isocyanatophenyl sulfide, bis(4-isocyanatophenyl)sulfide, bis(4-isocyanatomethylphenyl)sulfide and the like;

aromatic disulfide based polyisocyanate compounds such as bis(4-isocyanatophenyl)disulfide, bis(2-methyl-5-isocyanatophenyl)disulfide, bis(3-methyl-5-isocyanatophenyl)disulfide, bis(3-methyl-6-isocyanatophenyl)disulfide, bis(4-methyl-5-isocyanatophenyl)disulfide, bis(4-methoxy-3-isocyanatophenyl)disulfide and the like;

sulfur-containing alicyclic polyisocyanate compounds such as 2,5-diisocyanatotetrahydrothiophene, 2,5-diisocyanatomethyltetrahydrothiophene, 3,4-diisocyanatomethyltetrahydrothiophene, 2,5-diisocyanato-1,4-dithiane, 2,5-diisocyanatomethyl-1,4-dithiane, 4,5-diisocyanato-1,3-dithiolane, 4,5-bis(isocyanatomethyl)-1,3-dithiolane, 4,5-diisocyanatomethyl-2-methyl-1,3-dithiolane and the like;

aliphatic polyisothiocyanate compounds such as 1,2-diisothiocyanatoethane, 1,6-diisothiocyanatohexane and the like;

alicyclic polyisothiocyanate compounds such as cyclohexane diisothiocyanate and the like;

aromatic polyisothiocyanate compounds such as 1,2-diisothiocyanatobenzene, 1,3-diisothiocyanatobenzene, 1,4-diisothiocyanatobenzene, 2,4-diisothiocyanatotoluene, 2,5-diisothiocyanato-m-xylene, 4,4′-methylenebis(phenyl isothiocyanate), 4,4′-methylenebis(2-methylphenyl isothiocyanate), 4,4′-methylenebis(3-methylphenyl isothiocyanate), 4,4′-diisothiocyanatobenzophenone, 4,4′-diisothiocyanato-3,3′-dimethylbenzophenone, bis(4-isothiocyanatophenyl)ether and the like;

further, carbonyl polyisothiocyanate compounds such as 1,3-benzenedicarbonyl diisothiocyanate, 1,4-benzenedicarbonyl diisothiocyanate, (2,2-pyridine)-4,4-dicarbonyl diisothiocyanate and the like, sulfur-containing aliphatic polyisothiocyanate compounds such as thiobis(3-isothiocyanatopropane), thiobis(2-isothiocyanatoethane), dithiobis(2-isothiocyanatoethane) and the like;

sulfur-containing aromatic polyisothiocyanate compounds such as 1-isothiocyanato-4-[(2-isothiocyanato)sulfonyl]benzene, thiobis(4-isothiocyanatobenzene), sulfonyl(4-isothiocyanatobenzene), dithiobis(4-isothiocyanatobenzene) and the like, sulfur-containing alicyclic polyisothiocyanate compounds such as 2,5-diisothiocyanatothiophene, 2,5-diisothiocyanato-1,4-dithiane and the like; and

compounds having an isocyanato group and an isothiocyanate group such as 1-isocyanato-6-isothiocyanatohexane, 1-isocyanato-4-isothiocyanatocyclohexane, 1-isocyanato-4-isothiocyanatobenzene, 4-methyl-3-isocyanato-1-isothiocyanatobenzene, 2-isocyanato-4,6-diisothiocyanato-1,3,5-triazine, 4-isocyanatophenyl-4-isothiocyanatophenyl sulfide, 2-isocyanatoethyl-2-isothiocyanatoethyl disulfide and the like.

Furthermore, there can be used their halogen substituted compounds such as chlorine substituted compounds, bromine substituted compounds or the like, their alkyl substituted compounds, their alkoxy substituted compounds, their nitro substituted compounds, prepolymer type modified compounds modified with polyhydric alcohols, carbodiimide-modified compounds, urea-modified compounds, biuret-modified compounds, dimerization or trimerization reaction compounds or the like. These compounds may be used singly, or two or more compounds may be used in combination.

The proportion of the (poly)thiol compound and the polyiso(thio)cyanate compound is not particularly limited, but the molar ratio is usually in the range of 0.3 to 2.0 both inclusive (SH group/NCO group), preferably in the range of 0.7 to 2.0 both inclusive, and further preferably in the range of 0.7 to 1.3 both inclusive. When the proportion is within the above range, it is possible to satisfy each performance such as refractive index, heat resistance or the like which is desired as an optical material and a transparent material of a plastic lens with a good balance.

For purposes of improvement of general properties, operability, polymerization reactivity and the like of the polyurethane based resin of the present invention, other substances may be added, in addition to the ester compound and iso(thio)cyanate compound forming the urethane resin. For example, in addition to a urethane-forming starting material, one or two or more active hydrogen compounds having typical examples of amine and the like, epoxy compounds, olefin compounds, carbonate compounds, ester compounds, metals, metal oxides, organic metal compounds, inorganic substances or the like may be added.

Further, a variety of substances such as a chain extender, a crosslinking agent, a photostabilizer, a UV absorber, an antioxidant, an oil soluble dye, a filler, a releasing agent, and a blueing agent, may be added, depending on the purposes, as in a known molding method. In order to adjust to a desired reaction rate, a thiocarbamic acid S-alkyl ester or a known reaction catalyst used for producing polyurethane may be added as appropriate. The lens formed of the polyurethane resin of the present invention can be usually obtained by casting polymerization.

Specifically, the (poly)thiol compound obtained by the production process of the present invention is mixed with a polyiso(thio)cyanate compound to obtain a mixed solution containing the polymerizable composition of the present invention. This mixed solution is degassed according to a proper method as needed, and then injected into a mold and usually slowly heated from a low temperature to a high temperature for polymerization.

The thus-obtained polyurethane based resin of the present invention has a high refractive index, a low dispersion, excellent heat resistance and durability, light weight, and excellent impact resistance and the occurrence of whitening is further suppressed. Thereby it being suitable as an optical material and a transparent material for a spectacle lens, a camera lens, or the like.

Furthermore, the lens which is obtained by using the polyurethane resin of the present invention may be, if necessary, subjected to physical or chemical treatment such as surface abrasion treatment, antistatic treatment, hard coat treatment, non-reflective coat treatment, dyeing treatment and polarizing treatment, for prevention of reflection, enhancement of hardness, improvement of abrasion resistance, improvement of chemical resistance, supply of anticlouding, supply of fashionability, and the like.

### EXAMPLES

The present invention is now illustrated in detail below with reference to Examples. Thiourea in use, and the obtained (poly)thiol compound and the polyurethane based resin obtained by polymerization were analyzed in the following manner.
Content of calcium in thiourea: Thiourea was dissolved in water to give an aqueous solution, and then the calcium content was measured by an ion chromatographic method.
Color of polythiol (APHA: American Public Healthy Association): APHA was employed as an analyzing item for evaluating the color of the obtained (poly)thiol compound. APHA was measured in accordance with JIS K 0071-1. Specifically, APHA was obtained by comparing the color of a sample to diluted standard solution having an equivalent concentration using a standard solution prepared by melting a reagent of platinum and cobalt. Its degree was taken as a measurement value. The smaller the value was, the better the color was.
Color of polythiol (Y.I): Yellow index (Y.I.) was employed as an analyzing item for evaluating the color more in detail. Y.I. was measured by using a colorimeter CT-210 (a product of Minolta Co., Ltd.). Firstly, distilled water was fed into a cell CT-A20 having an optical path length of 20 mm, and a white calibration was performed as Y=100.00, x=0.3101 and y=0.3162. Thereafter, a sample was fed into the same cell and the color measurement was carried out. The measurement results, x and y values, were used to calculate Y.I. according to the following formula:

Y.I.=(234×x+106×y+106)/y (1)

This Y.I value was taken as a numerical value of the color of polythiol. The higher the numerical value was, the greater the coloring degree was.

When a liquid polythiol was measured, it was fed into a cell having a thickness of 10 mm for the measurement.
Color of polyurethane based resin (Y.I): A colorimeter, CT-210, manufactured by Minolta Co., Ltd. was used to measure the Y. I. of a plastic lens obtained from the polyurethane resin. A round flat plate with a thickness of 9 mm and φ of 75 mm was produced by cast polymerization, and then measured on the color coordination, x and y. Based on the resulting x and y values, the above equation (1) was used to determine the yellow index (Y. I).
Loss degree of transparency: As an analyzing item for evaluating the transparency of the plastic lens containing a polyurethane based resin, the loss degree of transparency was employed. The loss degree of transparency was obtained in the following means. The lens plate of a circular flat plate having a thickness of 9 mm and φ75 mm was prepared. Then, the lens plate was irradiated with a light source (Luminar Ace LA-150A, a product of Hayashi Watch Works Co., Ltd.) for measuring the loss degree of transparency with a gray scale image processing unit. Captured images were expressed in numbers by gray scale image processing to obtain the loss degree of transparency. When the loss degree of transparency is not more than 30, it was indicated with 0, while, when it was greater than 30, it was indicated with x.

### Reduction of Calcium Content in Thiourea

The calcium (Ca) content in thiourea was reduced by the following procedure.

Into a 2-liter, 4-necked flask equipped with a stirrer, a reflux condensing water separator, a nitrogen gas purge tube and a thermometer were introduced 1,530 weight parts of distilled water and 470.0 weight parts of thiourea with the purity of 98.2% containing Ca of 1.5 wt %. The resulting material was heated to 40 degree centigrade for removing an insoluble matter by filtering. Thereafter, the filtrate was cooled down to 5 degree centigrade, and thiourea was precipitated and crystallized at the same temperature for 3 hours. Thiourea was taken out by filtering, and vacuum-dried at 40 degree centigrade under 700 Pa to obtain 368.6 g of thiourea having a Ca content of 0.07 wt %.

Furthermore, in other Examples and Comparative Examples, the crystallization time was properly adjusted by using the aforementioned method to obtain various thioureas having different Ca contents.

### Example 1

### Synthesis of (poly)thiol compound having 1,2-bis[(2-mercaptoethyl)thio]-3-mercaptopropane as main ingredient

Into a 2-liter, 4-necked flask equipped with a stirrer, a reflux condensing water separator, a nitrogen gas purge tube and a thermometer were introduced 169 weight parts (2.16 mol) of 2-mercaptoethanol and 76.0 weight parts of water. At 30 degree centigrade, 91.9 weight parts (1.08 mol) of 47 wt % aqueous sodium hydroxide solution was added dropwise thereto over 30 minutes, and then 99.9 weight parts (1.08 mol) of epichlorohydrin was added dropwise at the same temperature over 3 hours, and the resulting solution was matured for 1 hour. Next, 450.0 weight parts (4.32 mol) of 35 wt % hydrochloric acid water and 246.9 weight parts (3.24 mol) of thiourea with the purity of 99.90% having a calcium content of 0.05 wt % obtained by recrystallization in advance were introduced, and the resulting solution was matured under reflux at 110 degree centigrade for 3 hours for producing a thiuronium salt. The solution was cooled down to 60 degree centigrade, and then 450.0 weight parts of toluene and 331.1 weight parts (4.86 mol) of 25 wt % aqueous ammonia solution were introduced thereinto for carrying out hydrolysis to obtain a toluene solution of polythiol having 1,2-bis[(2-mercaptoethyl)thio]-3-mercaptopropane as a main ingredient. The toluene solution was subjected to acid washing and water washing for removing toluene and a trace of water under heat and reduced pressure. Thereafter, 268.7 weight parts of polythiol having 1,2-bis[(2-mercaptoethyl)thio]-3-mercaptopropane as a main ingredient was obtained by filtering. APHA of the obtained polythiol was 10, while Y.I thereof was 0.70.

Production of Plastic Lens

52 weight parts of m-xylylene diisocyanate, 0.015 weight parts of dibutyltin dichloride as a curing catalyst, 0.10 weight part of Zelec UN (product name, acid phosphoric acid alkyl ester, a product of Stepan Co.) as an internal mold releasing agent and 0.05 weight parts of Viosorb 583 (product name, a product of Kyodo Chemical Co., Ltd.) as an ultraviolet absorber were mixed and dissolved at 20 degree centigrade. After mixing and dissolving were confirmed, subsequently into this mixed and dissolved solution was introduced 48 weight parts of polythiol having 1,2-bis[(2-mercaptoethyl)thio]-3-mercaptopropane as a main ingredient obtained as in the above, and the resulting solution was mixed to give a uniform mixed solution. This uniform solution was degassed at 600 Pa for 1 hour. Thereafter, the resulting solution was filtered using a 3-μm Teflon (registered trademark) filter, and then injected into a mold equipped with a glass mold and tapes. This mold was put into an oven and then gradually heated from 10 to 120 degree centigrade at which polymerization was conducted for 18 hours. After completion of polymerization, the mold was taken out from the oven and a resin was released from the mold. The obtained resin was additionally annealed at 120 degree centigrade for 3 hours. Y.I. of the obtained resin was 4.5 and the loss degree of transparency was 20. So, the evaluation was indicated with “∘” on the loss degree of transparency. The evaluation results are shown in Table 1.

### Example 2

Polythiol having 1,2-bis[(2-mercaptoethyl)thio]-3-mercaptopropane as a main ingredient was synthesized in the same manner as in Example 1, except that thiourea with the purity of 99.70% having a calcium content of 0.20 wt % obtained by recrystallization in advance was used instead of thiourea used in Example 1. APHA of the obtained polythiol having 1,2-bis[(2-mercaptoethyl)thio]-3-mercaptopropane as a main ingredient was 10, while Y.I. thereof was 0.81. Using this polythiol, a plastic lens was produced and evaluated in the same manner as in Example 1. The evaluation results of the obtained plastic lens are shown in Table 1.

### Example 3

Polythiol having 1,2-bis[(2-mercaptoethyl)thio]-3-mercaptopropane as a main ingredient was synthesized in the same manner as in Example 1, except that thiourea with the purity of 99.20% having a calcium content of 0.70 wt % obtained by recrystallization in advance was used instead of thiourea used in Example 1. APHA of the obtained polythiol having 1,2-bis[(2-mercaptoethyl)thio]-3-mercaptopropane as a main ingredient was 10, while Y.I. thereof was 0.93. Using this polythiol, a plastic lens was produced and evaluated in the same manner as in Example 1. The evaluation results of the obtained plastic lens are shown in Table 1.

### Example 4

Polythiol having 1,2-bis[(2-mercaptoethyl)thio]-3-mercaptopropane as a main ingredient was synthesized in the same manner as in Example 1, except that thiourea with the purity of 99.00% having a calcium content of 0.90 wt % obtained by recrystallization in advance was used instead of thiourea used in Example 1. APHA of the obtained polythiol having 1,2-bis[(2-mercaptoethyl)thio]-3-mercaptopropane as a main ingredient was 10, while Y.I. thereof was 0.95. Using this polythiol, a plastic lens was produced and evaluated in the same manner as in Example 1. The evaluation results of the obtained plastic lens are shown in Table 1.

### Example 5

### Synthesis of polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients

Into a 2-liter, 4-necked flask equipped with a stirrer, a reflux condensing water separator, a nitrogen gas purge tube and a thermometer were introduced 89.1 weight parts (1.14 mol) of 2-mercaptoethanol, 44.8 weight parts of water and 0.4 weight parts of 47 wt % aqueous sodium hydroxide solution. At 10 degree centigrade, 107.3 weight parts (1.16 mol) of epichlorohydrin was added dropwise over 4 hours, and the resulting solution was matured for 1 hour. Next, 261.6 weight parts (0.58 mol) of 16.9 wt % aqueous sodium sulfide solution was added dropwise thereto at 25 degree centigrade over 1 hour, and the resulting solution was matured at the same temperature for 3 hours. Subsequently, 211.8 weight parts (2.78 mol) of thiourea with the purity of 99.90% having a calcium content of 0.05 wt % obtained by recrystallization in advance was introduced, and the resulting solution was matured under reflux at 110 degree centigrade for 3 hours for producing a thiuronium salt. The solution was cooled down to 60 degree centigrade, and then 360.0 weight parts of toluene and 347.4 weight parts (5.10 mol) of 25 wt % aqueous ammonia solution were introduced thereinto for carrying out hydrolysis to obtain a toluene solution of polythiol having 4,8-dimercaptomethyl-1,11-dimercapto-3,6,9-trithiaundecane as a main ingredient. The toluene solution was subjected to acid washing and water washing for removing toluene and a trace of water under heat and reduced pressure. Thereafter, 198.8 weight parts of polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients was obtained by filtering. APHA of the obtained polythiol was 10, while Y.I. thereof was 1.20.

Production of Plastic Lens

50.7 weight parts of m-xylylene diisocyanate, 0.01 weight part of dibutyltin dichloride as a curing catalyst, 0.10 weight part of Zelec UN (product name, acid phosphoric acid alkyl ester, a product of Stepan Co.) as an internal mold releasing agent and 0.05 weight parts of Viosorb 583 (product name, a product of Kyodo

Chemical Co., Ltd.) as an ultraviolet absorber were mixed and dissolved at 20 degree centigrade. 49.3 weight parts of polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane obtained as in the above as main ingredients was introduced thereinto, and the resulting solution was mixed to give a uniform mixed solution. This uniform solution was degassed at 600 Pa for 1 hour. Thereafter, the resulting solution was filtered using a 3-μm Teflon (registered trademark) filter, and then injected into a mold equipped with a glass mold and tapes. This mold was put into an oven and then gradually heated from 10 to 120 degree centigrade at which polymerization was conducted for 18 hours. After completion of polymerization, the mold was taken out from the oven and a resin was released from the mold. The obtained resin was additionally annealed at 120 degree centigrade for 3 hours. Y.I. of the obtained resin was 5.0 and the loss degree of transparency was 23. So, the evaluation was indicated with “∘” on the loss degree of transparency.

### Example 6

Polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients was synthesized in the same manner as in Example 5, except that thiourea used in Example 2 was used instead of thiourea used in Example 5. APHA of the obtained polythiol was 10, while Y.I. thereof was 1.25. Using this polythiol, a plastic lens was produced and evaluated in the same manner as in Example 5. The evaluation results of the obtained plastic lens are shown in Table 1.

### Example 7

Polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients was synthesized in the same manner as in Example 5, except that thiourea used in Example 3 was used instead of thiourea used in Example 5. APHA of the obtained polythiol was 10, while Y.I. thereof was 1.33. Using this polythiol, a plastic lens was produced and evaluated in the same manner as in Example 4. The evaluation results of the obtained plastic lens are shown in Table 1.

### Example 8

Polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients was synthesized in the same manner as in Example 5, except that thiourea used in Example 4 was used instead of thiourea used in Example 5. APHA of the obtained polythiol was 10, while Y.I. thereof was 1.38. Using this polythiol, a plastic lens was produced and evaluated in the same manner as in Example 4. The evaluation results of the obtained plastic lens are shown in Table 1.

### Comparative Example 1

Polythiol having 1,2-bis[(2-mercaptoethyl)thio]-3-mercaptopropane as a main ingredient was synthesized in the same manner as in Example 1, except that thiourea with the purity of 98.70% having a calcium content of 1.20 wt % was used instead of thiourea used in Example 1. APHA of the obtained polythiol was 20, while Y.I. thereof was 2.01. Using this polythiol, a plastic lens was produced and evaluated in the same manner as in Example 1. The evaluation results of the obtained plastic lens are shown in Table 1.

### Comparative Example 2

Polythiol having 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients was synthesized in the same manner as in Example 5, except that thiourea with the purity of 98.70% having a calcium content of 1.20 wt % was used instead of thiourea used in Example 5. APHA of the obtained polythiol was 20, while Y.I. thereof was 2.10. Using this polythiol, a plastic lens was produced and evaluated in the same manner as in Example 5. The evaluation results of the obtained plastic lens are shown in Table 1.

TABLE 1

| | Evaluation Results | | | | |
|---|---|---|---|---|---|
| | | | | | Loss |
| | Calcium | | | | degree of |
| | amount in | | | Y.I. of | transparency |
| | thiourea | APHA of | Y.I. of | plastic | of plastic |
| | (wt %) | polythiol | polythiol | lens | lens |
| Example 1 | 0.05 | 10 | 0.70 | 4.5 | 20 (∘) |
| Example 2 | 0.20 | 10 | 0.81 | 4.7 | 22 (∘) |
| Example 3 | 0.70 | 10 | 0.93 | 4.8 | 23 (∘) |
| Example 4 | 0.90 | 10 | 0.95 | 5.0 | 26 (∘) |
| Example 5 | 0.05 | 10 | 1.20 | 5.0 | 23 (∘) |
| Example 6 | 0.20 | 10 | 1.25 | 5.3 | 26 (∘) |
| Example 7 | 0.70 | 10 | 1.33 | 5.4 | 28 (∘) |
| Example 8 | 0.90 | 10 | 1.38 | 5.5 | 29 (∘) |
| Comparative | 1.20 | 20 | 2.01 | 6.1 | 45 (x) |
| Example 1 | | | | | |
| Comparative | 1.20 | 20 | 2.10 | 6.8 | 50 (x) |
| Example 2 | | | | | |

From the above results, the (poly)thiol compounds obtained by using thiourea having a calcium content of not more than 1.0 wt % were excellent in the color, and the plastic lenses produced by using this (poly)thiol compound were also excellent in the color and transparency. On the other hand, in the (poly)thiol compounds obtained by using thiourea having a calcium content in excess of 1 wt % in Comparative Examples 1 and 2, the color was worsened, while in the obtained plastic lenses, the color and transparency were worsened, either. The resins obtained in Examples and Comparative Examples were all colorless and transparent when respective resins were viewed, but resins of Comparative Examples were observed as slightly yellow in comparison with resins of Examples when all resins were compared.

### INDUSTRIAL APPLICABILITY

According to the present invention, it is possible to produce a colorless and transparent (poly)thiol compound for an optical material in which coloring is suppressed, and a colorless and transparent (thio)urethane resin in which coloring and whitening are suppressed. The present invention greatly contributes to provision of optical materials and transparent materials, particularly plastic lenses for eyeglasses, in a stable manner.

1. A process for producing a (poly)thiol compound for an optical material comprising:
reacting an organic (poly)halogen compound or a (poly)alcohol compound with thiourea to produce an isothiuronium salt, and
hydrolyzing the obtained isothiuronium salt in the presence of ammonia water to produce a (poly)thiol compound,
in which the calcium content in the thiourea is not more than 1.0 wt %.

2. The process for producing a (poly)thiol compound for an optical material as set forth in claim 1, in which said (poly)thiol compound has a sulfur atom in addition to a thiol group.

3. The process for producing a (poly)thiol compound for an optical material as set forth in claim 2, in which said (poly)thiol compound having a sulfur atom in addition to a thiol group has one or two or more kinds selected from the group consisting of 1,2-bis[(2-mercaptoethyl)thio]-3-mercaptopropane, 4,8-dimercaptomethyl-1,11-dimercapto-3,6,9-trithiaundecane, 4,7-dimercaptomethyl-1,11-dimercapto-3,6,9-trithiaundecane and 5,7-dimercaptomethyl-1,11-dimercapto-3,6,9-trithiaundecane as main ingredients.

4. A polymerizable composition comprising the (poly)thiol compound for an optical material produced by the process as set forth in claim 1 and a polyiso(thio)cyanate compound.

5. A resin obtained by curing the polymerizable composition as set forth in claim 4.

6. An optical material comprising the resin as set forth in claim 5.

7. A lens comprising the resin as set forth in claim 5.
